(19) United States
(12) Patent Application Publication
(10) Pub. No.: US 2025/0320230 A1
(43) Pub. Date: Oct. 16, 2025

(54) STABLE ALKALI AMIDE SOLUTIONS AND PROCESS FOR THEIR PREPARATION

(71) Applicant: Albemarle Germany GMBH, Frankfurt am Main (DE)

(72) Inventors: Dirk DAWIDOWSKI, Friedberg (DE); Annika HÖRBERG, Mainz (DE); Uwe LISCHKA, Frankfurt am Main (DE); Theresa SCHMIDT, Frankfurt (DE); Johannes KLÖSENER, Kelkheim (DE); Ulrich WIETELMANN, Friedrichsdorf (DE); Peter RITTMEYER, Sulzbach/Taunus (DE); Stefan SCHNIPPERING, Siegburg (DE)

(73) Assignee: Albemarle Germany GMBH, Frankfurt am Main (DE)

(21) Appl. No.: 19/240,105

(22) Filed: Jun. 17, 2025

Related U.S. Application Data
(62) Division of application No. 17/258,842, filed on Jan. 8, 2021.

(30) Foreign Application Priority Data
Jul. 11, 2018 (DE) 10 2018 211 495.1

(51) Int. Cl. C07F 1/02

(52) U.S. Cl. CPC C07F 1/02

(57) ABSTRACT

The object of the invention are solutions of alkali metal amides MNR¹R², wherein M is an alkali metal selected from Li, Na, K, Rb, Cs; R¹ and R² independently of one another are linear, branched or cyclic alkyl groups having 1 to 8 C atoms or together are a cycloalkyl radical, the alkali metal amides being present in methyltetrahydropyran or in a solvent mixture containing methyltetrahydropyran, and processes for their preparation.

### CROSS-REFERENCE TO RELATED APPLICATIONS

This application is a divisional of U.S. application Ser. No. 17/258,842, filed Jan. 8, 2021, entitled “STABLE ALKALI AMIDE SOLUTIONS AND PROCESSES FOR PREPARING SAME” which is a 371 application of PCT/EP2019/067477, filed Jun. 28, 2019, entitled “STABLE ALKALI AMIDE SOLUTIONS AND PROCESSES FOR PREPARING SAME” which claims priority to German Application No. 10 2018 211 495.1 filed Jul. 11, 2018, entitled “STABLE ALKALI AMIDE SOLUTIONS AND PROCESSES FOR PREPARING SAME”, the entire contents of which are incorporated herein by reference.

### PRIOR ART

Ether-based solvents are among the most important industrially used process solvents. In numerous organic reactions they are used as solvents due to their good solubility and donor effect.

Besides diethyl ether, cyclic tetrahydrofuran has become established as one of the most important aprotic donor solvents for reactions using saline, basic and organometallic reagents. It has good dissolving properties and is considered to be largely inert. However, ether cleavage must be expected when using stronger nucleophiles.

Therefore, solutions of strongly nucleophilic organometallic compounds such as butyllithium in THF are not stable (see M. Schlosser, Organometallics in Synthesis, John Wiley, 1994, 130, or R. B. Bates, J. Org. Chem., 37(4), 1972, 560). Even metal amide bases that are weakened in their nucleophilicity, such as lithium diisopropylamide (LDA), have a limited shelf life as solutions because they decompose under THF cleavage, among other things:

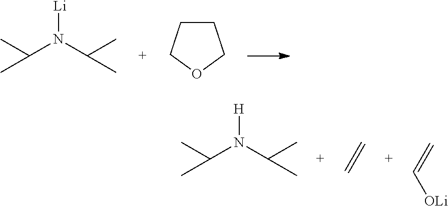

At storage temperatures of 23 or 40° C., they decompose at rates of 0.1 or 0.3% per day (see “Lithium & Magnesium Amides”, Technical Brochure of Albemarle 10/2016).

It is also known that base metals such as alkali metals can also react with ether solvents. It is known that the lithium/THF system is not thermodynamically stable, but tends to dangerous decomposition reactions. In 2001, a serious incident occurred at a Swiss company, caused by an uncontrollable decomposition reaction of lithium residues with THF (see Annual Report 2001 of the Safety Inspectorate of the Canton of Basel-Landschaft, 2002/40-10, pages 12-14). DSC (dynamic differential calorimetry) tests show that the system consisting of lithium powder and THF reacts exothermically depending on the lithium concentration from 100-120° C. (“Tₒₙₛₑₜ”). The decomposition energy released in this process is in the range between 60-80 kJ/mol lithium (see Lithium Topics, September 2002, Technical Information of Chemetall GmbH). According to the general safety standards (see J. Barton, R. Roberts, Chemical Reaction Hazards, Institution of Chemical Engineers, 1997, ISBN 0 95295 341 0, or Th. Grewer's, Thermal Hazards of Chemical Reactions, Industrial Safety Series, Vol. 4, Elsevier 1994, ISBN 0-444-89722-4), the synthesis temperature must not exceed the value of Tonset-100 K, in this case 0-20° C. On an industrial scale, these low temperatures require a high energy input for cooling and long dosing and reaction times, which in turn leads to correspondingly high production costs.

### Problem to be Solved

The problem to be solved by the invention is to provide alkali amide solutions, in particular alkali metal dialkylamides in donor solvents and methods for the safe and inexpensive preparation of strongly basic reagents.

### Solution of the Problem

The problem is solved by solutions of alkali metal amides MNR¹R², wherein M is an alkali metal selected from Li, Na, K, Rb, Cs; R¹ and R² are independently linear, branched or cyclic alkyl groups with 1 to 8 C atoms or together they are a cycloalkyl radical, wherein the alkali metal amides being present in a methyltetrahydropyran or in a methyltetrahydropyran containing solvent mixture.

Preferably M is lithium and R¹ and R² are isopropyl groups. The strongly basic reagent is then lithium diisopropylamide (LDA).

It is particularly preferred that the solvent mixture contains, in addition to 4-methyltetrahydropyran, at least one hydrocarbon which is liquid at room temperature and the molar ratio between the alkali metal amide LDA and 4-methyltetrahydropyran is preferably between 1:0.5 and 1:3.

According to the invention, the preparation is carried out in methyltetrahydropyran or in a solvent mixture containing methyltetrahydropyran, i.e., instead of THE, a solvent with a similarly good solubility but significantly improved stability against alkali metals and metal dialkylamides is used. The molar ratio between metal dialkylamide and methyltetrahydropyran is at least 0.5:1, preferably at least 1:1. A methyltetrahydropyran (MTHP), particularly preferably 4-methyltetrahydropyran (4-MTHP), is used as such solvent. 4-MTHP boils at 105° C. and has only a very low solubility in water (1.5%). In comparison with water-miscible THF, the low water solubility allows convenient product isolation by phase separation processes and the reduction of wastewater quantities. 4-MTHP possesses with μ=about 1.864 D (product information from Kuraray, 17.08.2017) a slightly higher dipole moment than tetrahydrofuran with μ=1.75 D (see D. R. Lide, Handbook of Organic Solvents, CRC Press Boca Raton, 1995, ISNN: 0-8493-8930-5).

Surprisingly, it was found that methyltetrahydropyrans, especially 4-MTHP, have a significantly improved thermal stability against alkali metals and alkali amide bases. This effect comes as a surprise to the expert because methyltetrahydropyrans have at least comparable polarity and thus a similarly strong donicity as THF and contain the same structural elements, i.e. CO bonds that are in principle susceptible to bases, in a ring system.

R¹ and R² are preferably selected from the group consisting of methyl, ethyl, n-propyl, iso-propyl, n-butyl, sec-butyl, isobutyl, tert-butyl, pentyl, neopentyl, hexyl, octyl, decyl, 2-ethylhexyl, cyclopentyl, cyclohexyl. Cyclic amides are preferably metal piperidides, especially the metal 2,2,6,6-tetramethylpiperidide. The metal is preferably selected from lithium and sodium.

In a preferred embodiment, only methyltetrahydropyrans, especially only 4-methyltetrahydropyran, are used as solvents.

Mixtures of at least one methyltetrahydropyran and at least one hydrocarbon solvent are particularly preferred as solvents.

Preferably, aliphatic, cycloaliphatic or aromatic compounds are used as hydrocarbons, either alone or as a mixture of several hydrocarbons.

The hydrocarbon used is at least one compound selected from the group consisting of pentanes, hexanes, heptanes, octanes, decanes, cyclohexane, methylcyclohexane, benzene, toluene, ethylbenzene, xylenes and/or cumene.

As a mixture of several hydrocarbons, the commercially available products, i.e. boiling cuts, are preferably used.

In addition, if a hydrogen acceptor is used for the synthesis, the alkali amide solution according to the invention contains the partially hydrogenated form of this hydrogen acceptor in the amount determined by the synthesis stoichiometry. The hydrogenated hydrogen acceptor is, for example, ethylbenzene when styrene is used or 2-methyl-2-butene when isoprene is used.

The alkali metal is preferably used in powder form or as granules with grain sizes <100 μm or <10 mm, respectively.

Furthermore, the molar ratio between alkali metal and methyltetrahydropyran is preferably 1:0.5 to 1:3.

It is advantageous to add at least one hydrogen acceptor A selected from the group consisting of α-aryl olefins or 1,3-diene with 4 to 12 C atoms during the synthesis reaction.

Especially preferred the hydrogen acceptor A is selected from the group consisting of: styrene, a-methylstyrene, butadiene, isoprene or 1,3-cyclohexadiene and is preferably used in a molar ratio of 0.3 to 0.6:1, based on the amount of amine used.

A process in which the reaction temperature is between 0 and 200° C., preferably 20 to 110° C., is particularly preferred.

The improved stability properties are first demonstrated using the ether solvent/lithium metal system as an example.

For this purpose thermochemical investigations were carried out with the RADEX system of the company Systag/Switzerland. Mixtures of 0.09 g lithium metal powder with a particle size <100 μm and 1.8 g ether solvent were filled into steel autoclaves under argon blanket gas and heated up to a final temperature of 250° C. While the mixture of Li and THF decomposes strongly exothermically from a temperature of about 80° C. (peak temperature 150° C., heat of decomposition −150 J/g), no exotherm is observed in the case of the mixture with 4-MTHP. Instead, a weak endotherm is registered at a furnace temperature of about 190° C., which can be attributed to the melting of the metallic lithium. This means that the lithium metal/4-MTHP system is much more stable and thus allows correspondingly higher process temperatures when lithium and other alkali metals are used.

Surprisingly, similar effects were also observed in the case of mixtures of alkali metal amides with the ethers under consideration. Corresponding measurement results for solutions of lithium diisopropylamide (LDA) in the two ethers are summarized in Table 1. In the case of the THF solutions (comparative tests) two consecutive exothermic events are observed, in the case of the 4-MTHP solutions only one exothermic decomposition step is observed. It can be seen that the beginning of the decomposition reaction (TONSET) in 4-MTHP is shifted about 90 K to higher temperatures. In addition, the heat of decomposition is significantly lower.

TABLE 1
Thermal stability (Radex tests) of LDA solutions
in the solvent mixture ether/heptane

| | Molar ratio | LDA | | | | |
|---|---|---|---|---|---|---|
| Donor | LDA/donor | concentration | Tₒₙₛₑₜ | | | Decomposition |
| solvent | solvent | (weight %) | (° C.) | T_{max.-1} | T_{max.-2} | heat (J/g) |
| THF | 1.1 | 20 | 103 | 128 | 170 | n.b. |
| THF | 1.3 | 20 | 100 | 135 | 174 | −64 |
| 4-MTHP | 1.0 | 21 | 190 | 200 | ./. | −31 |
| 4-MTHP | 1.5 | 22 | 193 | 204 | ./. | −29 |

The improved thermal stability also affects the storage temperature with a mild temperature increase. This is shown in Table 2:

TABLE 2
Storage stability of LDA solutions in
the solvent mixture ether/heptane

| | Molar ratio | LDA | Decomposition rate (% | |
|---|---|---|---|---|
| Donor | LDA/donor | concentration | per day) at | |
| solvent | solvent | (weight %) | 20° C. | 40° C. |
| THF | 2.0 | 28 | −0.15 | −1.01 |
| THF | 1.1 | 20 | −0.1 | −0.4 |
| 4-MTHP | 1.5 | 20 | −0.02 | −0.04 |

The effects shown on the example of the LDA are also transferable to other alkali metal amides MNR¹R². The preferred alkali metals are M=lithium, sodium or potassium. Alkali amides are produced in a similar way to the process known from the use of THF (see U.S. Pat. No. 4,595,779): Either by direct reaction of the alkali metal with a secondary amine in a solvent or solvent mixture containing methyltetrahydropyran according to:

M+H-NR¹R²>MNR¹R²+½H₂

or preferably from the alkali metal and a secondary amine in the presence of a hydrogen acceptor A according to:

2 M+2 H-NR¹R²+A>2 MNR¹R²+AH₂

where M is an alkali metal selected from Li, Na, K, Rb, Cs; R¹ and R² are independently linear, branched or cyclic alkyl groups having from 1 to 8 carbon atoms or together represent a cycloalkyl radical. The hydrogen acceptor A represents an-arylolefin or a 1,3-diene with 4 to 12 C-atoms. R¹ and R² are particularly preferred and independently of each other: methyl, ethyl, n-propyl, isopropyl, n-butyl, sec-butyl, isobutyl, tert-butyl, pentyl, neopentyl, hexyl, octyl, decyl, 2-ethylhexyl, cyclopentyl, cyclohexyl. As cyclic amines, piperidine and 2,2,6,6-tetramethylpiperidine are preferred. The preferred hydrogen acceptors are styrene, a-methylstyrene, butadiene, isoprene and 1,3-cyclohexadiene. The hydrogen acceptor is preferably used in a molar ratio of 0.3 to 0.6:1 relative to the amount of amine used. The alkali metal is preferably used either in powder form or as granules with particle sizes <100 μm or <10 mm, respectively. A pure methyltetrahydropyran, preferably pure 4-methyltetrahydropyran, can be used as solvent. However, preferably mixtures of a hydrocarbon, which is liquid at room temperature, and a methyltetrahydropyran are used. The hydrocarbon can be aliphatic, cycloaliphatic or aromatic compounds, either in pure form or as a mixture. Particularly preferred aliphates are: pentanes, hexanes, heptanes, octanes and decanes, preferred cycloaliphatics are cyclohexane and methylcyclohexane, preferred aromatics are benzene, toluene, ethylbenzene, xylenes and cumene. The mixing ratio between methyltetrahydropyran and hydrocarbons can be between 10:1 and 1:10 (the numbers represent weight ratios). The molar ratio between alkali metal and methyltetrahydropyran is preferably 1:0.5 to 1:3.

Due to the improved thermal stability and safety with regard to an undesired reaction with the solvent associated with the use of methyltetrahydropyrans, the reaction can also be carried out at higher temperatures using liquid alkali metals. The latter process is preferred especially for the low melting alkali metals sodium, potassium, rubidium and cesium.

The reaction temperature is generally between 0 and 200° C., preferably 20 to 110° C.

The alkali metal amide solutions prepared by a process according to the invention in a solvent or hydrocarbon solvent mixture containing methyltetrahydropyran preferably have a concentration of at least 0.3 mol/kg, particularly preferred at least 0.5 mol/kg. The molar ratio between the alkali metal amide and the methyltetrahydropyran is preferably between 1:0.5 and 1:3. A solution of lithium diisopropylamide (LDA) in a mixture of at least one methyltetrahydropyran and at least one hydrocarbon is particularly preferred.

The process products manufactured in accordance with the invention are used as bases in organic synthesis, for example for selective enolization.

The invention is explained in more detail using an example and two figures.

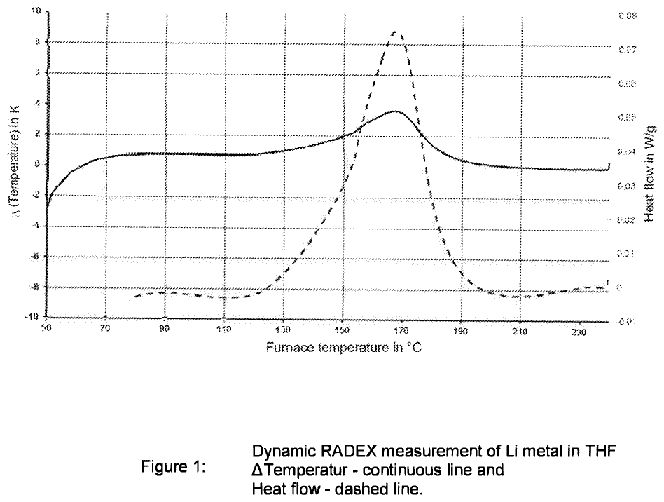

FIG. 1 shows: The thermal behavior (Radex test) of a mixture of 0.09 g Li-powder and 1.8 g THF

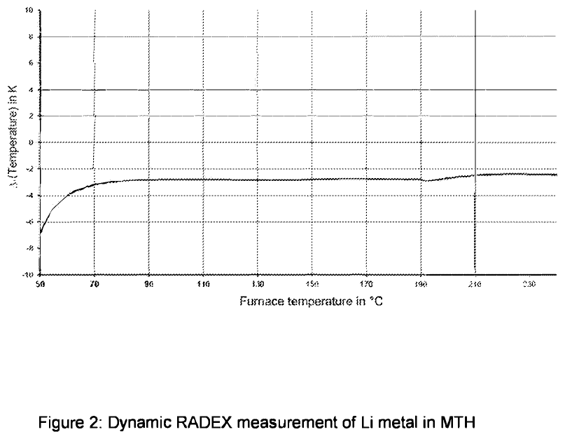

FIG. 2 shows: The thermal behavior (Radex test) of a mixture of 0.09 g Li powder and 1.8 g 4-MTHP

### Example 1

Preparation of a 20% solution of lithium diisopropylamide (LDA) in 4-methyltetrahydropyrane/heptane/ethylbenzene

107 g heptane (isomer mixture “Iparsol 7”, available from DHC GmbH), 79 g 4-MTHP (supplier Kuraray), 3.45 g lithium granulate (edge length about 3 mm, available from Albemarle) and 50.4 g diisopropylamine (supplier Merck) were placed in an inert, i.e. dry 500 ml glass reactor filled with the blanket gas argon and stirred for one hour at 25° C. Then it was heated up to 30° C. and 26.2 g styrene were added from a dropping funnel within three hours. During the addition, the mixture discolored slightly brownish.

After the end of dosing, the turbid reaction mixture was stirred for another 30 minutes and then filtered through a glass frit. The result was 251 g of a clear yellowish product solution. The active base concentration (thermometry method) was 1.82 mmol/g, resulting in an LDA concentration of 19.5 wt %.

1. A method for the preparation of alkali metal amides MNR¹R², wherein M is an alkali metal selected from Li, Na, K, Rb, and Cs; R¹ and R² independently of one another are linear, branched or cyclic alkyl groups having 1 to 8 C atoms or together are a cycloalkyl radical,
characterized in that the preparation is carried out in a solvent,
wherein the solvent is a mixture containing 4-methyltetrahydropyran and at least one aromatic hydrocarbon compound selected from one or more of benzene, toluene, ethylbenzene, xylene, and/or cumene, and
wherein the molar ratio between alkali metal amide and 4-methyltetrahydropyran is at least 0.5:1.

2. The method according to claim 1, characterized in that R¹ and R² are selected from the group consisting of methyl, ethyl, n-propyl, isopropyl, n-butyl, sec-butyl, isobutyl, tert-butyl, pentyl, neopentyl, hexyl, octyl, decyl, 2-ethylhexyl, cyclopentyl, cyclohexyl, and a common cycloalkyl radical having 3 to 12 C atoms.

3. The method according to claim 2, characterized in that R¹ and R² are 2,2,6,6-tetramethylpiperidide.

4. The method according to claim 1, characterized in that the alkali metal is used in powder form or as granules with grain sizes <100 μm or <10 mm, respectively.

5. The method according to claim 1, characterized in that the molar ratio between alkali metal and 4-methyltetrahydropyran is 1:0.5 to 1:3.

6. The method according to claim 1, characterized in that at least one hydrogen acceptor A is present, and wherein the at least one hydrogen acceptor A is selected from the group consisting of α-aryl olefins, and 1,3-diene having 4 to 12 C atoms.

7. The method according to claim 6, characterized in that the hydrogen acceptor A is selected from the group consisting of styrene, a-methylstyrene, butadiene, isoprene, and 1,3-cyclohexadiene.

8. The method according to claim 6, characterized in that the hydrogen acceptor is used in a molar ratio of 0.3:1 to 0.6:1, based on the amount of amine used, and the reaction temperature being between 0° C. and 200° C.

9. The method according to claim 6, characterized in that the reaction temperature being between 20° C. and 110° C.

10. The method according to claim 1, characterized in that R¹ and R² are 2,2,6,6-tetramethylpiperidide.

11. The method according to claim 1, characterized in that the at least one aromatic hydrocarbon compound is ethylbenzene.

12. The method according to claim 1, characterized in that a mixing ratio between the 4-methyltetrahydropyran and the at least one aromatic hydrocarbon compound is between 10:1 and 1:10.
